(19) United States
(12) Patent Application Publication
(10) Pub. No.: US 2020/0008905 A1
(43) Pub. Date: Jan. 9, 2020

(54) DENTAL SPLINT AND METHOD FOR PRODUCING A DENTAL SPLINT

(71) Applicants: Steffen G. Tschackert, Frankfurt am Main (DE); Peiker Holding GmbH, Bad Homburg v. d. Hohe (DE)

(72) Inventors: Lutz RICHTER, Mixdorf (DE); Steffen G. TSCHACKERT, Frankfurt am Main (DE); Christoph SCHILLO, Friedrichsdorf (DE)

(73) Assignees: Peiker Holding GmbH, Bad Homburg v. d. Hohe (DE); Steffen G. Tschackert, Frankfurt am Main (DE)

(21) Appl. No.: 16/575,818

(22) Filed: Sep. 19, 2019

Related U.S. Application Data
(63) Continuation of application No. PCT/EP2018/084657, filed on Dec. 13, 2018.

(30) Foreign Application Priority Data
Dec. 14, 2017 (DE) 10 2017 129 957.2

(51) Int. Cl. A61C 5/00; A61B 5/00; A61B 5/0205; A61B 5/01; A61B 5/145; B33Y 80/00; B33Y 10/00; B29C 64/194

(52) U.S. Cl. CPC A61C 5/007; A61B 5/0002; A61B 5/02055; A61B 5/01; A61B 5/021; B33Y 80/00; B33Y 10/00; B29C 64/194; A61B 5/14532

(57) ABSTRACT

The invention relates to a dental splint as an assembly comprising a first subassembly embodied as a dental splint body and having a surface that comes into contact with an oral cavity of a user, a second subassembly embodied as electronics, at least one third subassembly embodied as a sensor, a fourth subassembly embodied as an energy source, at least one fifth subassembly embodied as an antenna, and a sixth subassembly embodied as electrical connection means, wherein the second to fifth subassemblies are connected by the sixth subassembly. In this case, at least one of the second to sixth subassemblies is produced in each case completely or partly in a 3-D printing method.

### CROSS REFERENCE TO RELATED APPLICATIONS

This application is a continuation of International Application No. PCT/EP2018/084657 filed Dec. 13, 2018, which designated the United States, and claims the benefit under 35 USC § 119(a)-(d) of German Application No. 10 2017 129 957.2 filed Dec. 14, 2017, the entireties of which are incorporated herein by reference.

### FIELD OF THE INVENTION

The present invention relates to a dental splint, and a method for producing a dental splint.

### BACKGROUND OF THE INVENTION

DE 10 2004 043 665 A1 discloses a dental splint comprising a first subassembly embodied as a dental splint body and having a surface that comes into contact with an oral cavity of a user, a second subassembly embodied as electronics, at least one third subassembly embodied as a sensor, a fourth subassembly embodied as an energy source, at least one fifth subassembly embodied as an antenna, and a sixth subassembly embodied as electrical connection means, wherein the second to fifth subassemblies are connected by the sixth subassembly.

Furthermore, US 2014/0072926 A1 discloses a dental splint produced by means of a 3-D printer.

### SUMMARY OF THE INVENTION

It is an object of the present invention to propose a dental splint and a method for producing a dental splint, which dental splint is producible in a compact design and cost-effectively despite numerous subassemblies and which method is producible in a compact design and cost-effectively despite the presence of numerous subassemblies.

In the case of the dental splint according to the present invention, at least one of the second to sixth subassemblies is produced in each case completely or partly in a 3-D printing method. As a result, a compact design can be realized by virtue of a flat implementability of structures produced using 3-D printing. Furthermore, creating at least one part of at least one of the second to sixth subassemblies or at least one of the second to sixth subassemblies in the 3-D printing method greatly reduces the manufacturing outlay and thus saves costs since cost-intensive handling is obviated in the case of printed structures.

Furthermore, it is provided that the antenna comprises at least one antenna conductor track and/or the electrical connection means comprise at least one current conductor track, which are/is embodied in the dental splint body below the surface thereof and produced in the 3-D printing method. Such conductor tracks can be produced in a simple manner by means of 3-D printing methods. A further advantage is that even a complex course of the conductor tracks is producible without high additional outlay and the conductor tracks extend in a protected manner in the dental splint body.

Alternatively, it is also provided that the antenna comprises at least one antenna conductor track and/or the electrical connection means comprise at least one current conductor track, which are/is embodied as a part of the surface of the dental splint body and produced in the 3-D printing method. As a result, the transmission and reception behavior of the antenna is influenced by the dental splint body to a lesser extent, such that improved transmission and reception properties of the antenna are achieved. Current conductor tracks routed at the surface of the dental splint body can be contacted in a simple manner, such that contact with the dental splint e.g. for the purpose of charging the energy store is possible in a cost-effective manner.

Furthermore, it is provided that the antenna conductor track and/or the current conductor track have/has in the direction of the extent thereof a bent course and/or a change in the cross-sectional shape thereof and/or the cross-sectional area thereof. As a result, the antenna conductor track and/or the current conductor track can easily be adapted to a topography predefined by the dental splint body and can easily be optimized to the respective requirements.

It is also provided that at least one of the sensors comprises at least one probe, wherein the probe is embodied as a probe conductor track produced in the 3-D printing method, which probe conductor track is embodied, in particular, in such a way that it extends through the dental splint body to the surface of the dental splint body and either forms with an end section a part of the surface or projects by the end section beyond the surface, in particular, for making contact with a mucous membrane. As a result, probes of sensors can be produced in a simple manner since handling of miniature components is not required.

Provision is furthermore made for embodying the antenna conductor track and/or the current conductor track and/or the probe conductor track as an electrical conductor and the dental splint body as an insulator. As a result, the antenna conductor track and/or the current conductor track and/or the probe conductor track can be laid in the dental splint body without a dedicated insulating layer.

It is also provided that the dental splint body embodied as an insulator is constructed from a first plastic, and, in particular, from the first plastic and at least one second plastic different than the first plastic in terms of at least one material property, in particular, a degree of hardness, in a 3-D printing method, wherein provision is made, in particular, for the first plastic, by which the dental splint bears against molars, to have a lower degree of hardness than the second plastic, by which the dental splint bears against incisors. As a result, the wearing comfort of the dental splint can be improved, such that the user perceives the dental splint as less disturbing.

It is also provided that at least one cavity is formed for at least one of the second to fifth subassemblies in the dental splint body depending on the site of use thereof, in particular, lingually (near the mandible) or palatally (near the maxilla), wherein the at least one cavity is produced by an additive manufacturing method, such as, in particular, a 3-D printing method, or by a subtractive method, such as, in particular, a milling method or drilling method. Appropriate accommodation disturbs the user to a lesser extent in terms of his/her feeling in the oral cavity, such that the dental splint obtains significantly higher acceptance for long wearing times of a number of hours.

Furthermore, provision is made for embodying the sensor as a blood sugar sensor and/or as a pressure sensor and/or as a temperature sensor and/or as a gyrosensor and/or as an acceleration sensor and/or as a blood pressure sensor and/or as a heart rate sensor. By virtue of such sensors, the dental splint is usable for a multiplicity of applications in the medical field, in everyday life and in sports.

Furthermore, the present invention provides for the energy source to be embodied as an energy converter and, in particular, as a thermoelectric energy converter and/or as a kinetic energy converter, which converts kinetic energy into electrical energy. As a result, the dental splint becomes an autonomous component which can be worn even over relatively long periods of time.

Finally, it is provided that the second subassembly and/or the third subassembly and/or the fourth subassembly and/or the fifth subassembly and/or the sixth subassembly are/is produced in each case completely or partly in the 3-D printing method. As a result, the dental splint can be realized to an optimum extent by 3-D printing.

In the method according to the present invention for producing a dental splint which is embodied, in particular, as claimed in at least one of the preceding claims and which comprises a first subassembly embodied as a dental splint body, a second subassembly embodied as electronics, at least one third subassembly embodied as a sensor, a fourth subassembly embodied as an energy source and at least one fifth subassembly embodied as an antenna, and a sixth subassembly embodied as electrical connection means, the steps mentioned below are provided:

3-D printing of the dental splint body,

interrupting the 3-D printing of the dental splint body at least once by means of a printing pause before completing the dental splint body,

carried out in an arbitrary order or in parallel in the at least one printing pause or in different printing pauses:
placing at least one of the second to sixth subassemblies in the region of an area of the dental splint body that is to be printed still further, and/or complete or partial 3-D printing of at least one second to sixth subassembly in the region of an or the area of the dental splint body that is to be printed still further,

printing to completion the dental splint body embodied as a first subassembly with complete or partial embedding of the second to sixth subassemblies.

As a result of the at least one printing pause when creating the dental splint body, the complete dental splint comprising all subassemblies can be produced in a comparatively simple manner as an assembly in which the second to sixth subassemblies are optimally accommodated or integrated in the first subassembly, such that the assembly is suitable for use in the oral cavity.

It is also provided that the 3-D printing of at least one of the second to sixth subassemblies, namely, in particular, the second subassembly and/or the third subassembly and/or the fourth subassembly and/or the fifth subassembly and/or the sixth subassembly, is carried by means of a second 3-D printing method, which differs from a first 3-D printing method used for printing the dental splint embodied as a first subassembly, wherein the two different 3-D printing methods are chosen, in particular, from the group of the methods mentioned below:

3-D printing using powder (3DP), in particular selective laser sintering (SLS) or electron beam melting (EBM) or electron beam additive manufacturing (EBAM),

3-D printing by means of fused materials, in particular, “Fused Filament Fabrication” (FFF) or fused deposition (e.g. FDM—Fused Deposition Modeling),

3-D printing using liquid materials, in particular stereolithography (STL, SLA) or “Digital Light Processing” (DLP) or “Multi Jet Modeling” (MJM) or “Polyjet” methods or “Film Transfer Imaging” methods (FTI). The use of different 3-D printing methods makes it possible to produce a component that is optimized with regard to the, in some instances different, production engineering requirements of all the subassemblies.

Finally, it is provided that before and after the printing pause for the 3-D printing of the first subassembly a first material is used, having different material properties than a second material used for the 3-D printing in the printing pause, wherein the first material is electrically insulating, in particular, and wherein the second material is electrically conductive, in particular. As a result, a dedicated insulation of the electrically conductive structures can be dispensed with since this insulation are embedded and thus insulated by the further construction of the dental splint body that proceeds after the printing pause.

### BRIEF DESCRIPTION OF THE DRAWINGS

Further details of the present invention are described on the basis of schematically illustrated exemplary embodiments in the drawing, in which:

FIG. 1 shows a schematic illustration of one embodiment variant of a dental splint according to the present invention in perspective view.

### DETAILED DESCRIPTION OF THE INVENTION

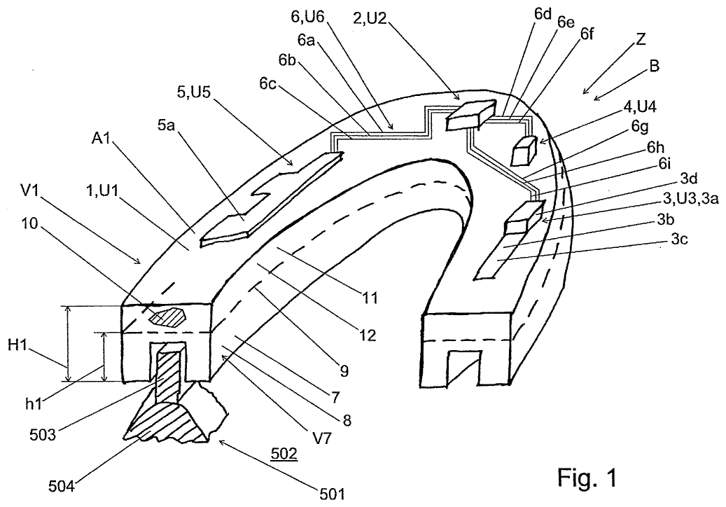

FIG. 1 shows a schematic illustration of one embodiment variant of a dental splint Z according to the present invention in perspective view. The dental splint Z is embodied as an assembly B and comprises six subassemblies U1 to U6.

A dental splint body 1 forms the first subassembly U1 and its surface A1 is in contact with an oral cavity 502 of a user 501 in the case of use. Of the oral cavity 502, a molar 503 and gum 504 surrounding the latter are shown here merely by way of example and schematically in FIG. 1. The second subassembly U2 is formed by electronics 2. The third subassembly U3 is formed by a sensor 3. The fourth subassembly U4 is formed by an energy source 4. The fifth subassembly U5 is formed by an antenna 5. The sixth subassembly U6 is formed by electrical connection means 6.

The electrical connection means 6, proceeding from the electronics 2, connect the electronics 2 to the third subassembly U3, the fourth subassembly U4 and the fifth subassembly U5 in a star-shaped fashion. An energy supply of the electronics 2 by the energy source 4 is ensured as a result. An energy supply of the sensor 3 via the electronics 2 and a communication between the sensor 3 and the electronics 2 are also ensured as a result. An exchange of transmission and reception signals between the antenna 5 and the electronics 2 is furthermore possible as a result.

The antenna 5 comprises an antenna conductor track 5a and the electrical connection means 6 comprise a multiplicity of current conductor tracks 6a to 6c, 6d to 6f and 6g to 6i. All five subassemblies U2 to U6 are embedded into a volume V1 of the dental splint body 1 below the surface A1 of the subassembly U1, such that they are arranged without contact with the oral cavity 502. In this case, the sensor 3 is embodied as a temperature sensor 3a in order to monitor a user's body temperature. For this purpose, the sensor 3 comprises a probe 3b embodied in the form of a probe conductor track 3c.

In accordance with an embodiment variant that is not illustrated, provision is also made for embodying the sensor as a blood sugar sensor comprising at least one probe which, for analyzing saliva situated in the oral cavity, projects beyond the surface of the dental splint body or forms a part of the surface of the dental splint body.

The first embodiment variant of the dental split Z as shown in FIG. 1 is produced in such a way that the dental splint body 1 or the first subassembly U1 is firstly printed from an electrically insulating or electrically non-conductive material up to a first height h1 in a 3-D printing method, thereby creating a lower part 7 of the subassembly U1. A volume V7 of this lower body 8 formed by the lower part 7 is indicated by dashed lines 9 in FIG. 1. Afterward, the electronics 2, the energy source 4 and a sensor body 3d of the sensor 3 are placed on an area 10 of said lower body 8 that is to be printed further, and are adhesively connected thereto. Cumulatively and alternatively, it is also provided that the area 10 that is to be printed further has one or more cavities produced by the 3-D printing process or produced after the 3-D printing process by means of a subtractive manufacturing method, such as e.g. drilling or milling, into which cavity or cavities the electronics 2, the energy source 4 and the sensor body 3d of the temperature sensor 3a are inserted. Before or after the placement or insertion, the antenna conductor track 5a, the current conductor tracks 6a to 6c, 6d to 6f and 6g to 6i and the probe conductor track 3c are printed by means of an electrically conductive printing material in a further 3-D printing method. Afterward—namely after the printing pause for producing the dental splint body 1—for the purpose of embedding the subassemblies U1 to U6, further 3-D printing with the insulating material is then carried out, wherein an upper body 12 of the dental splint body 1, the upper body being embodied as an upper part 11 of the subassembly U1, is printed, the upper body extending from the first height h1 to a second height H1 of the dental splint body 1, such that the dental splint Z has been completely produced upon the conclusion of this 3-D printing process.

The surface A1 of the dental splint body 1 or of the dental splint Z is illustrated as a transparent surface A1 in FIG. 1. Accordingly, FIG. 1 should be understood such that the second to sixth subassemblies U2 to U6 are arranged in the volume V1 of the dental splint body 1.

### LIST OF REFERENCE SIGNS:

1 Dental splint body
2 Electronics
3 Sensor 3
3a Temperature sensor
3b Probe of 3a
3c Probe conductor track of 3a
3d Sensor body of 3a
4 Energy source
5 Antenna
5a Antenna conductor track of 5
6 Electrical connection means
6a-6i Current conductor track of 6
7 Lower part of the subassembly U1
8 Lower body 8
9 Dashed lines 9
10 Area of 8 that is to be printed further
11 Upper part of the subassembly U1
12 Upper body of the dental splint body 1
501 User
502 Oral cavity
503 Molar
504 Surrounding gum
A1 Surface of 1
B Assembly
h1 First height of 1
H1 Height
U1-U6 Subassembly U1 to U6
V1 Volume of the dental splint body 1
V7 Volume of 7
Z Dental splint Z

1. A dental splint as an assembly comprising:
a first subassembly embodied as a dental splint body and having a surface that comes into contact with an oral cavity of a user,
a second subassembly embodied as electronics, at least one third subassembly embodied as a sensor, a fourth subassembly embodied as an energy source, at least one fifth subassembly embodied as an antenna, and a sixth subassembly embodied as an electrical connection,
wherein the second to fifth subassemblies are connected by the sixth subassembly,
wherein at least one of the second to sixth subassemblies is produced at least partly by a 3-D printing method, and
wherein the dental splint body is embodied as an insulator constructed from a first plastic and at least one second plastic different than the first plastic in terms of at least one material property in a 3-D printing method.

2. The dental splint as claimed in claim 1, wherein the antenna comprises at least one antenna conductor track and/or the electrical connection comprises at least one current conductor track, which are/is embodied in the dental splint body below the surface thereof and produced by a 3-D printing method.

3. The dental splint as claimed in claim 1, wherein the antenna comprises at least one antenna conductor track and/or the electrical connection comprises at least one current conductor track, which are/is embodied as a part of the surface of the dental splint body and produced by a 3-D printing method.

4. The dental splint as claimed in claim 2, wherein the antenna conductor track and/or the current conductor track have/has in the direction of the extent thereof
a bent course and/or
a change in the cross-sectional shape thereof and/or the cross-sectional area thereof.

5. The dental splint as claimed in claim 2,
wherein the sensor comprises at least one probe embodied as a probe conductor track produced by a 3-D printing method,
which probe conductor track is embodied such that it extends through the dental splint body to the surface of the dental splint body and either forms with an end section a part of the surface of the dental splint body or projects by the end section beyond the surface of the dental splint body for making contact with a mucous membrane.

6. The dental splint as claimed in claim 5, wherein the antenna conductor track and/or the current conductor track and/or the probe conductor track are/is embodied as an electrical conductor and the dental splint body is embodied as an insulator.

7. The dental splint as claimed in claim 1, wherein the first plastic, by which the dental splint bears against molars, has a lower degree of hardness than the second plastic, by which the dental splint bears against incisors.

8. The dental splint as claimed in claim 1, wherein at least one cavity is formed for at least one of the second to fifth subassemblies in the dental splint body depending on the site of use thereof, wherein the at least one cavity is produced by an additive manufacturing method or by a subtractive method.

9. The dental splint as claimed in claim 1, wherein the sensor is embodied as a blood sugar sensor and/or as a pressure sensor and/or as a temperature sensor and/or as a gyrosensor and/or as an acceleration sensor and/or as a blood pressure sensor and/or as a heart rate sensor.

10. The dental splint as claimed in claim 1, wherein the energy source is embodied as a thermoelectric energy converter and/or as a kinetic energy converter.

11. The dental splint as claimed in claim 1, wherein the second subassembly and/or the third subassembly and/or the fourth subassembly and/or the fifth subassembly and/or the sixth subassembly are/is produced in each case at least partly by a 3-D printing method.

12. A method for producing a dental splint which is embodied as claimed in claim 1, comprising:
3-D printing of the dental splint body,
interrupting the 3-D printing of the dental splint body at least once by a printing pause before completing the dental splint body,
carrying out in an arbitrary order or in parallel in the at least one printing pause or in different printing pauses:
placing at least one of the second to sixth subassemblies in the region of an area of the dental splint body that is to be printed still further, and/or at least partial 3-D printing of at least one second to sixth subassembly in the region of an or the area of the dental splint body that is to be printed still further,
printing to completion the dental splint body embodied as a first subassembly with at least partial embedding of the second to sixth subassemblies.

13. The method as claimed in claim 11, wherein the 3-D printing of at least one of the second to sixth subassemblies is carried by a second 3-D printing method, which differs from a first 3-D printing method used for printing the dental splint embodied as a first subassembly, wherein the two different 3-D printing methods are chosen from the following group:
3-D printing using powder and selective laser sintering or electron beam melting or electron beam additive manufacturing,
3-D printing using fused materials, and Fused Filament Fabrication or Fused Deposition Modeling,
3-D printing using liquid materials and stereolithography or Digital Light Processing or Multi Jet Modeling or Polyjet methods or Film Transfer Imaging methods.

14. The method as claimed in claim 11, wherein before and after the printing pause for the 3-D printing of the first subassembly a first material is used, having different material properties than a second material used for the 3-D printing in the printing pause, wherein the first material is electrically insulating and wherein the second material is electrically conductive.
